SEQUENCE LISTING (7 sequences)

SEQ ID NO: 1 - RNA, 42 nt, Artificial Sequence
  Description of Artificial Sequence Synthetic oligonucleotide
ucaccauuacuagguuugugauuaaaccuaguaauggugcuc

SEQ ID NO: 2 - RNA, 42 nt, Artificial Sequence
  Description of Artificial Sequence Synthetic oligonucleotide
uguugagucagagcuaugugauuauagcucugacucaaccuc

SEQ ID NO: 3 - RNA, 42 nt, Artificial Sequence
  Description of Artificial Sequence Synthetic oligonucleotide
ucauuaguaggguugaugugauuaucaacccuacuaaugcuc

SEQ ID NO: 4 - RNA, 42 nt, Artificial Sequence
  Description of Artificial Sequence Synthetic oligonucleotide
uucacuguacacucgaugugauuaucgaguguacagugacuc

SEQ ID NO: 5 - RNA, 42 nt, Artificial Sequence
  Description of Artificial Sequence Synthetic oligonucleotide
uuuacacauuagggcuugugauuaagcccuaauguguaacuc

SEQ ID NO: 6 - RNA, 42 nt, Artificial Sequence
  Description of Artificial Sequence Synthetic oligonucleotide
uacgugacacguucggugugauuaccgaacgugucacgucuc

SEQ ID NO: 7 - DNA, 229 bp, Severe acute respiratory syndrome coronavirus 2
caatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

(19) United States
(12) Patent Application Publication
(10) Pub. No.: US 2022/0145290 A1
(43) Pub. Date: May 12, 2022

(54) Substrate Sequences for the RNAi-Mediated Regulation of Genomic and Sub-genomic Viral RNAs

(71) Applicant: Speratum Biopharma, Inc., Dover, DE (US)

(72) Inventors: Christian Roberto Marin-Muller, San Jose (CR); Osvaldo Vega Martinez, Heredia (CR); Juan Carlos Valverde-Hernandez, Alajuela (CR)

(73) Assignee: Speratum Biopharma, Inc., Dover, DE (US)

(21) Appl. No.: 17/401,139

(22) Filed: Aug. 12, 2021

Related U.S. Application Data
(60) Provisional application No. 63/064,446, filed on Aug. 12, 2020.

(51) Int. Cl. C12N 15/11; G06F 17/16; C12Q 1/6809

(52) U.S. Cl. CPC C12N 15/11; G06F 17/40; C12Q 1/6809; G06F 17/16

(57) ABSTRACT

Provided is a method for identifying an RNAi sequence, comprising the steps of screening for one or more 7-mers from a gRNA; selecting a first number of the one or more 7-mers; characterizing one or more hit sites for each of the one or more 7-mers; calculating a hit per genomic region for each of the 7-mers; selecting a second number of the 7-mers; creating a frequency matrix for one or more 15-mers associated to each of the 7-mers; generating one or more generated 15-mers for each of the 7-mers; characterizing hits in the gRNA for each of the one or more generated 15-mers; calculating a summary index based on a cumulative hit frequency and a length; and selecting the RNAi sequences based on one or more generated features and one or more structural features.

### CLAIM OF PRIORITY

This application claims priority from U.S. Provisional Patent Application No. 63/064,446, filed on Aug. 12, 2020, the contents of which are incorporated herein by reference.

### FIELD OF THE INVENTION

The invention is related generally to the field of RNAi sequences. More particularly, the invention relates to apparatuses, methods, and systems for RNAi sequence identification.

### INTRODUCTION

Coronaviruses, and in particular SARS-CoV-2, have caused a viral outbreak leading to a worldwide pandemic of Covid-19 illness. Current circumstances globally have resulted in a worldwide public health emergency.

As a human pathogen, according to the World Health Organization (WHO), as of August 2020, over eighteen (18) million cases of Covid-19 have been confirmed globally, with at least 687,000 deaths. By August 2021, the number of global cases and deaths has risen to over two hundred (200) million cases, with at least four (4) million deaths.

It is now known and understood that SARS-CoV-2 is readily transmitted from human to human, spreading to multiple continents and leading to the WHO's declaration of a Public Health Emergency of International Concern (PHEIC) on 30 Jan. 2020.

While scientists have made progress in developing possible new treatments, efficacy and safety of such highly-experimental treatments remain unproven, and many questions remain unanswered. Though vaccines or other antiviral treatments for SARS-CoV-2 are of the greatest necessity, still many lack effectiveness.

While efficacy is critical to treating the SARS-CoV-2 pandemic, such treatments must also be shown to be safe. Indeed, smaller safety trials must be run to demonstrate safety before engaging in widespread efficacy trials, resulting in additional time.

It would be desirable, therefore, to provide treatments with increased chance of efficacy and safety, in order to maximize the likelihood of success and potential time to approval for treatment of SARS-CoV-2.

### SUMMARY

In an embodiment, the invention of the present disclosure may be a method for identifying an RNAi sequence, comprising the steps of identifying a potential 7-mer sequence; and registering one or more hit sites, where the one or more hit sites are one or more positions in a genomic RNA sequence where the potential 7-mer sequence is present. In an embodiment, the method may further comprise the steps of registering, alongside with the one or more hit sites, a name of the genomic region where the 7-mer sequence was found and a registered 15-mer sequence starting at a 7-mer sequence start position; generating, using the registered 15-mer sequence, a nucleotide position frequency matrix and a generated 15-mer for each 7-mer sequence based on the most frequent nucleotide for each position; and predicting, using the generated 15-mer sequence, one or more hits for a putative RNAi molecule. In an embodiment, the method may also comprise the steps of calculating a hit feasibility index for the one or more registered hit sites; generating a general index; and developing a summary matrix.

In an embodiment, the potential 7-mer sequence may be identified by screening for the most 7-mers in a target RNA. In an embodiment, the one or more hits for the putative RNAi molecule may be predicted based on perfect matching. In an embodiment, the hit feasibility index may assign a weight to each hit site based on a match sequence ΔG value and an AU content proportion 30 nucleotides upstream and downstream from the hit site. The hit feasibility index may be calculated via the following equation: Hₙ_{ₘ}=log₃(p(AU)ₙ_{ₙ}×−ΔGₙ_{ₘ}), where n is the generated 15-mer used to predict a m hit site, H is the hit feasibility, ΔG is the free energy required for the match to happen considering only base pairing, and p(AU) is the proportion 30 nt upstream and downstream from the hit site.

In an embodiment, the general index may be calculated by the following equation: IGₘ=Σₖ₌₁(Σₘ₌₁ Hₙ_{ₘ}), where IG is the general index which summarizes the effect of the hits, and A is the amount of hits for the n generated 15-mer of the match. The summary matrix may comprise the 7-mer, the 7-mer's generated 15-mer, a sense strand, the RNAi proposed sequence, the hit feasibility index, and the general index. In a further embodiment, the summary matrix may comprise a proposed antisense strand. In yet another embodiment, the summary matrix may further comprise a seed sequence GC content and a guide strand GC content.

In an embodiment, the invention of the present disclosure may be a method for identifying an RNAi sequence, comprising the steps of screening for one or more 7-mers from a gRNA; selecting a first number of the one or more 7-mers, the first number based on the frequency of the one or more 7-mers; and characterizing one or more hit sites for each of the one or more 7-mers. The method may further comprise the steps of calculating a hit per genomic region for each of the one or more 7-mers; selecting a second number of the one or more 7-mers, the second number based on the most hits in a sgRNA; and creating a frequency matrix for one or more 15-mers associated to each of the one or more 7-mers. In a further embodiment, the method also includes the steps of generating, based on the most frequent nucleotides, one or more generated 15-mers for each of the one or more 7-mers; characterizing hits in the gRNA for each of the one or more generated 15-mers; calculating a summary index based on a cumulative hit frequency and a length; and selecting a third number of the RNAi sequences based on one or more generated features and one or more structural features.

In an embodiment, the first number, the second number, and the third number may be any appropriate quantities. In an embodiment, the first number may be 500. In an embodiment, the second number may be 50. In an embodiment, the third number may be 5. In an embodiment, the one or more generated features and the one or more structural features may be a function of the summary index.

### BRIEF DESCRIPTION OF THE DRAWINGS

FIG. 1 illustrates an embodiment of an RNAi design workflow, in accordance with the invention.

FIG. 2A is a table depicting five RNAi SARS-CoV-2 silencing sequences.

FIG. 2B is a table depicting the number of matching sites of RNAi SARS-CoV-2 silencing sequences.

FIG. 3A is a table of sequences that may attack SARS-CoV-2 RNA.

FIG. 3B is a depiction of 3′UTR (untranslated region) of the SARS-CoV-2 RNA.

FIG. 4 is a graph of NPM-SC2 sequences 3′UTR regulation.

FIG. 5 is a graph of NPM-SC2 sequences cytotoxicity.

### DETAILED DESCRIPTION

Provided herein are novel treatments for SARS-CoV-2. In an embodiment, ribonucleic acid (RNA), such as RNA-interference (RNAi) molecules are utilized. The RNAi molecules may be used for the downregulation of viral RNA translation.

Small-interfering RNAs (siRNAs) may be designed to bind to specific target sequences. Off-target effects may occur through non-specific binding, and present a limitation to siRNA design. MicroRNAs (miRNAs) are naturally occurring RNAi molecules. They differ from siRNAs in that a miRNA may have evolved to target multiple different targets simultaneously, through virtue of imperfect pairing. This imperfect pairing leads to differential regulatory responses that differ from the canonical siRNA regulation. In accordance with an embodiment, the RNAi molecules described here are designed as synthetic molecules in a manner similar to siRNAs, but taking into account the permissibility of imperfect pairing as with a miRNA. Thus, the antiviral oligonucleotides generated provide broader, more flexible targeting, and can anticipate off-target effects.

SARS-CoV-2 is found in the order Nidoviridae, due to its unique, nested replication strategy of 30 kb or similarly-sized single-stranded viral RNA is processed into many smaller messenger RNAs (mRNAs), each encoding multiple proteins. The SARS-CoV-2 mRNAs contain a 3′UTR, the targeting sites remain intact even once the nested RNA replication process has begun. Thus, in accordance with the invention, an RNAi-based attack against viral RNA is proposed, to target such nested sequences.

In accordance with the invention, proposed are siRNA and miRNA sequences including, but not limited to, one or more of: strand-specific RNA sequences (ssRNA); non-canonical processing; no passenger strands; target minimization in human genome in order to maximize targets in viral genome; administrable with any suitable nucleic acid delivery vehicle; elevated silencing rates, short processing routes to bioactive states; multiple targeting sites to reduce genetic variability between strains; simplified production and isolation/purification in comparison to standard duplexes, and insertable into alternative siRNA construct design.

In accordance with principles of the invention, a genomic understanding of SARS-CoV-2 may be used to develop drugs and treatments tailored to treating the novel coronavirus associated with Covid-19. Thus, viral RNA of SARS-CoV-2 may be targeted with an anti-SARS-CoV-2 treatment: siRNA.

In an embodiment, the siRNA is specifically designed with the ability and potential to act on multiple target sites on the RNA of SARS-CoV-2. In an embodiment, in particular, the siRNA is designed to act on a plurality of sites on (1) the 3′UTR region of mRNA, and (2) the coding region (CDS) of SARS-CoV-2 RNA. Due to its location immediately after the translation termination codon, 3′UTR mRNA may be responsible for regulating gene expression, and are therefore well suited for receiving a therapeutic. By targeting such sites with siRNA, Ago2 mediated-cleavage may occur, which may result in the destruction of viral RNA prior to ribosome entry, thereby resulting in reduction of viral replication of SARS-CoV-2. Alternatively, inhibition of viral RNA translation may occur, through repression of ribosome drop-off or sequestering of the viral RNA, all through interaction with the target sequences.

RNA viruses are likely to mutate, and the use of RNAi-inducing molecules against such viruses may result in imperfect matches, due to the specific complementarity needed for siRNA activity. Moreover, siRNAs tend to require integrity of a relatively long sequence, which may make mutations even more cumbersome, in order to appropriately match with an intended target RNA.

Accordingly, the invention contemplates addressing likely mutations and requirements for high complementarity of siRNA through the generation of a synthetic oligonucleotide that behaves like a miRNA. Often, miRNA is less specific, and more suitably, may match with a greater number of match sites per target RNA. As such, due to increased promiscuity for regulatory target sites, miRNA regulation to specific RNA targets may be better suited for genetic variability and mutation, as compared to siRNA targeting. Due to the presence of only one match site, siRNA complementary site deletion eliminates the siRNA's silencing effect on the target RNA, increasing the likelihood of mutation destroying its regulation. In contrast, miRNAs, with multiple complementary sites on target, are capable of maintaining regulation even when one match site is deleted or changed. In an embodiment, however, a single target site with the right sequence may be sufficient for effective regulation.

Therefore, in an embodiment, a proprietary RNAi is provided, specifically formulated to increase silencing specificity while maximizing the number of complementary sites in the selected RNA target. As such, RNA-based viruses are able to be treated effectively, even with their high mutation rates. Accordingly, in accordance with numerous embodiments, the invention provides systems and methods identifying potential targeting sites, and provides systems and methods for designing target sequences, in order to maximize antiviral targeting, while decreasing the risk of mutation and target destruction. In some embodiments, combinations of RNAi molecules can be used, with either miRNA or siRNA-designed molecules, or may be used alone. The RNAi molecules may target any or multiple portions of the viral genome.

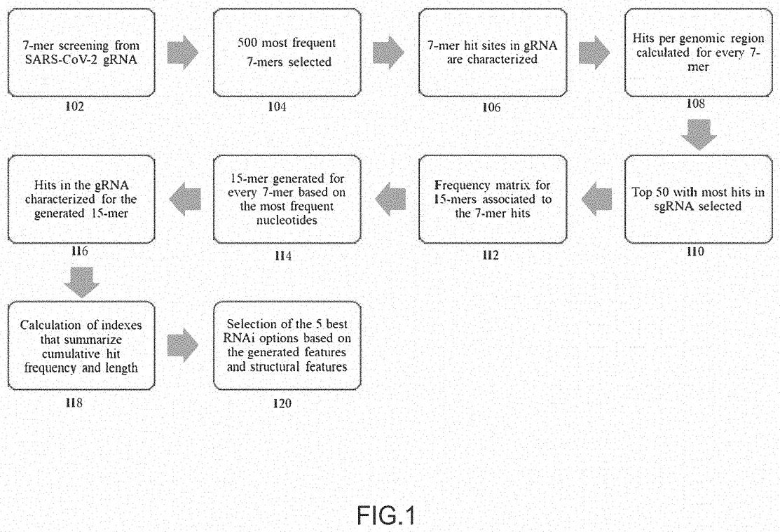

Referring to FIG. 1, FIG. 1 illustrates a non-limiting example of an RNAi design workflow, in accordance with the invention. At a first step 102, identification of potential RNAi sequences is performed (for example, the 7-mer screening from SARS-CoV-2 gRNA). In a second step 104, a potential 7-nucleotide seed sequence may be searched for, by searching the most frequent 7-mers in the target RNA (for example, the 500 most frequent 7-mers selected). As a non-limiting example, 5′UTR and CDS may be evaluated, due to the RNA-induced silencing process causing a silencing of transcripts through regulation in 5′UTR and CDS. In an embodiment, after compiling the 7′ mers, in step 104, the 500 most frequent ones may be selected for further analysis.

In a next step 106, every position in the genomic RNA sequence where the selected 7-mers are present, known as the hit site, may be registered alongside with the position, the name of the genomic region where the 7-mer was found, and the 15-mer starting at the 7-mer start position. For example, the 7-mer hit sites in gRNA may be characterized. Fifty 7-mers with the most hits regions may be selected. In step 108, the hits per genomic region may be calculated for every 7-mer. In step 110, the top fifty with the most hits in sgRNA may be selected.

In step 112, using the registered 15-mers, a nucleotide position frequency matrix may be generated, and a 15-mer may be generated. In step 114, each 7-mer is based on the most frequent nucleotide for each position, with the sequence corresponding to the proposed sense strand for the RNAi. Accordingly, non-canonical base paring and beyond seed sequence pairing may be promoted.

In step 116, hits of generated 15-mer sequences may be predicted for the presumed RNAi molecule based on perfect matching. Thus, perfect seed sequence and beyond seed sequence matches may be searched. For example, matches from 6 to 22 nucleotides long where registered. Although a large body of research supports and validates the effect of 6, 7 and 8 nucleotide long matches, shorter RNAi matches with transcripts such as 5 nucleotide long match and larger matches such as 10 and 15 matches may be shown to be involved in the silencing of RNA molecules.

In step 118, for every registered hit site, a hit feasibility index may be calculated. A weight may be assigned to every hit, based on its match sequence ΔG value and the AU content proportion 30 nt upstream and downstream from the hit site. A calculation may be performed of indexes that summarize cumulative hit frequency and length. In step 120, a selection of the 5 best RNAi options may be made based on the generated features and structural features.

Thus, in those embodiments where the structure of the seed sequence is highly responsible for its target silencing capacities, lower ΔG seed sequence pairing values tend to correlate with higher suppression levels.

Where higher AU contents are found near the RNAi binding sites, this may be correlated with more transcript and protein silencing, alluding to usually a less stable secondary structure.

Hₙ_{ₘ}=(p(AU)ₙ_{ₘ}×ΔGₙ_{ₘ})

Disclosed above is an exemplary equation where n is the generated 15-mer used to predict the m hit site, H is the hit feasibility, ΔG is the free energy required for the match to happen considering only base pairing and is the p(AU) proportion 30 nt upstream and downstream from the hit site. Next, two indexes may be calculated for the generated 15-mers:

$${IG}_{m} = {\sum }_{k = 1}^{15} ( {\sum }_{m = 1}^{A} {H}_{{n}_{m}} )$$

In an embodiment, IG is the general index which summarizes the effect of the hits, A is the amount of hits for the n generated 15-mer of the match.

Referring to step 112, a matrix may be developed featuring: the 7-mer, it's generated 15-mer or proposed antisense strand, the sense or guide strand, the RNAi proposed sequence, the seed sequence GC content, the guide strand GC content, as well as all aforementioned indexes. In an embodiment, human transcripts that may be silenced by seed off-target effects may be searched using the Custom microRNA Prediction functionality of the miRDB platform.

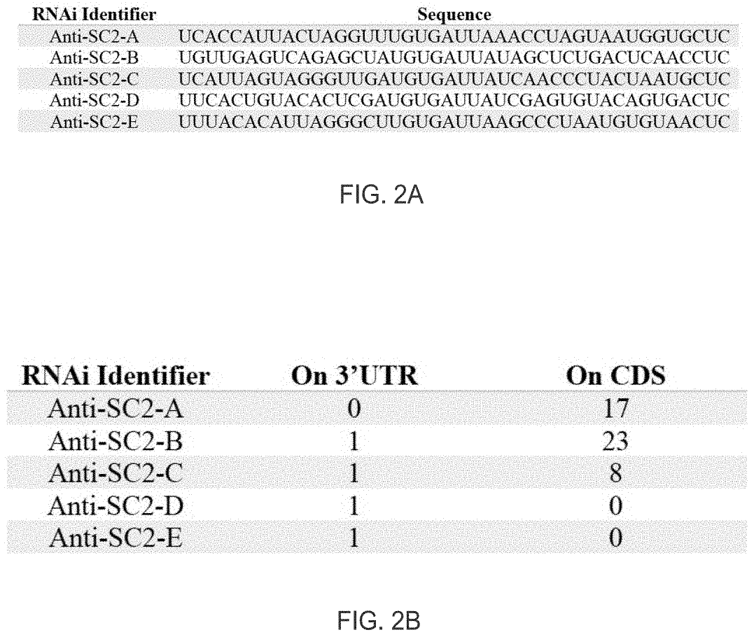

Referring to FIG. 2A, the table depicts five RNAi SARS-CoV-2 silencing sequences, in accordance with an embodiment

Referring to FIG. 2B, the table depicts the number of matching sites of RNAi SARS-CoV-2 silencing sequences.

As disclosed herein, the invention may be modified by chemical modifications, as contemplated. Alternatively, molecular presentation may be modified to administer treatment as an RNAi mimic or as an expression vector, such as a virus, plasmid, or others.

The various embodiments may be modified in combination with various delivery methods, such as LNP, polymeric nanoparticles, aptamer associated delivery, antibody delivery, affimer associated delivery or metal nanoparticles.

Further, treatment may be administered either as linear or circular RNA, or as part of a longer non-coding RNA. Treatment may also be administered as a DNA counterpart, including via a plasmid or other system vector, or shRNA vector system.

The embodiments disclosed herein combine the benefits of siRNA and miRNA to add capacity to have multiple complementarity sites, as in miRNA, while promoting a high specificity similar to siRNA. Additionally, this may address issues of mutation, and may be used to design RNAi molecules to target either single or multiple selected RNA.

Therefore, the various embodiments may be designed to silence coding and non-coding RNA or silence single-selected RNA or combinations of RNA. Further, regions inside the RNA to be targeted may be enriched for target sites. These embodiments may be implemented clinically or non-clinically, and administered alone or in combination with other compounds.

In an embodiment, there may be a number of SARS-CoV-2 in vitro surrogate expression methods.

Regarding a cell culture, in an embodiment, ACE2 and TMPRSS2-expressing human colon carcinoma cell-line Caco-2 (ATCC® HTB-37™), human embryonic kidney cell-line HEK 293T (ATCC® CRL-3216™) and grivet kidney cell-line Vero E6 (ATCC® CRL-1586™) may be maintained in DMEM medium (D6429, Sigma-Aldrich) supplemented with 10% fetal bovine serum (F2442, Sigma-Aldrich), antibiotic-antimycotic solution (A5955, Sigma-Aldrich), and 1× of non-essential amino acid solution (M714, Sigma-Aldrich).

Regarding, plasmid construct design, in an embodiment, for a first approach of the therapeutic potential of the anti SARS-CoV-2 designed miRNA mimics, a Firefly-Renilla luciferases reporter vector and a S-protein/N-protein expression vector may be designed as follows: (a) For the Firefly-Renilla luciferases reporter vector, the SARS-CoV-2 universal 3′UTR sequence may be synthetized and cloned in the 3′ end of the Firefly luciferase transcript of the pmirGLO Vector (E1330, Promega) by external services (GenScript). The Renilla luciferase transcript may not include any SARS-CoV-2 elements, being the endogenous control; and (b) For the S-protein/N-protein expression vector, the SARS-CoV-2 S-protein and N-protein (1) mRNA complete sequences and (2) 3′UTR paring sequences mutated mRNA may be synthetized and cloned in the phMGFP vector (E6421, Promega) by external services (GenScript). These designs may allow the discrimination between 3′UTR and 5′UTR-CDS regulation capabilities of the anti-SARS-CoV-2 designed miRNA mimics.

Regarding transfection, in an embodiment, Lipofectamine3000 (L3000015, ThermoFisher) may be be used for transient expression of the plasmid constructs on the Caco-2, HEK 293T and Vero E6 cell-lines following manufacturer instructions.

Regarding a luciferase assay, in an embodiment, the assays may be performed using the Dual-Glo® Luciferase Assay System (E2940, Promega) following manufacturer instructions.

Regarding eukaryotic expression assays, in an embodiment, specifically RT-qPCR, the total RNA may be extracted from cell-lines by using the mirVana™ miRNA Isolation Kit (AM1561, ThermoFisher) per manufacturer instructions and, RT-qPCR may be carried out by using the SuperScript™ III One-Step RT-PCR System kit (12574026, ThermoFisher) per manufacturer instructions in a Q qPCR machine (Quantabio).

Regarding a western blot, in an embodiment, the total protein may be extracted from cell-lines by using RIPA Buffer (R0278, Sigma-Aldrich), Protease Inhibitor Tablets (S8820, Sigma-Aldrich) and Protease Inhibitor Cocktail (P8340, Sigma-Aldrich) following the recommended protocol. Total protein quantification may be done by using Pierce™ BCA Protein Assay Kit (23227, Thermo Fisher) per manufacturer instructions.

Regarding evaluation of viral titer in SARS-CoV-2 in vitro model using live virus, in an embodiment, SARS-CoV-2 (accession: NC 045512.2) stock may be be cultured following conditions previously described. Virus stocks will be titrated on Vero E6 cells (ATCC® CRL-1586™), in a biosafety level-4 (BSL-4) facility. Virus titration assay may be done following the protocol previously described. Virus titter may be calculated using the formula: PFU/mL=number of plates counted/(inoculation volume (50 uL in this case)*sample dilution).

For example, this work may be be performed through external services or collaborations. RT-qPCR may be used to confirm viral RNA loads and the specific identity of SARS-CoV-2. Standard and approved methodologies for amplification of viral RNA may be used.

Regarding the dose-range finding safety study in an outbred murine model, CD-1 mice (CD-1® IGS Mice, Charles River) may be used as experimental subjects. Mice may be grouped according to their weight. The experimental subjects may be treated intravenously every other day with a determined dosing amount in a final injection volume of 200 μL for one month. Once the treatment regimen is completed, blood parameters including, but not limited to, reference factors for hepatic and kidney functions, may be evaluated as the safety reference. Pathology analysis for various body tissues may be performed by a certified veterinary pathologist.

Regarding the efficacy study in a transgenic murine or other animal model, in an embodiment, a novel transgenic mouse model may be developed for studying SARS-CoV-2. In such a case, specific pathogen-free, 6-11-month-old, female wild type (WT) C57BL/6J (000664) and, transgenic (TG) K18-hACE2 (034860) mice may be obtained. In an embodiment, two experiments may be run: (a) Nanoparticle formulation delivery: SARS-CoV-2 free WT and TG mice may be dosed intravenously in a q.o.d regimen (M, W, F) with the nanoparticle formulation containing an Anti-SARS-CoV-2 miRNA mimic. After completion of treatment, mice may be sacrificed, and Anti-SARS-CoV-2 miRNA mimic may be quantified on the respiratory system by RT-qPCR; and (b) Efficacy study: TG mice may be inoculated intranasally with SARS-CoV-2 at a dosage of 10⁵ TCID₅₀. In an embodiment, after confirmed infection, mice will be separated in two groups: (1) Intravenously dosing of the nanoparticle formulation containing an Anti-SARS-CoV-2 miRNA mimic in a q.o.d regimen (M, W, F) and, (2) Intravenously dosing of the nanoparticle formulation containing an scramble miRNA mimic in a q.o.d regimen (M, W, F). Animals may be continuously observed daily to record body weights, clinical symptoms, responsiveness to external stimuli and death.

However, if the aforementioned model is not available or an alternative animal model is deemed more appropriate, an adequate alternative research methodology may be proposed.

Regarding human blood immune response ex vivo assay, in an embodiment, to determine whether the formulated nanoparticle induces a human cytokine response, it may be incubated for 24 hours in whole blood collected from healthy volunteers. Anticoagulant may be added to the whole blood to prevent coagulation and the formulated nanoparticle may be tested at three different concentrations. After the incubation, the plasma may be separated by centrifugation and IL-1β, IL-6, IL-12, INFα, and TNFα may be analyzed by ELISA (KHC0011, BMS223HS, KHC0121, BMS213HS, ThermoFisher).

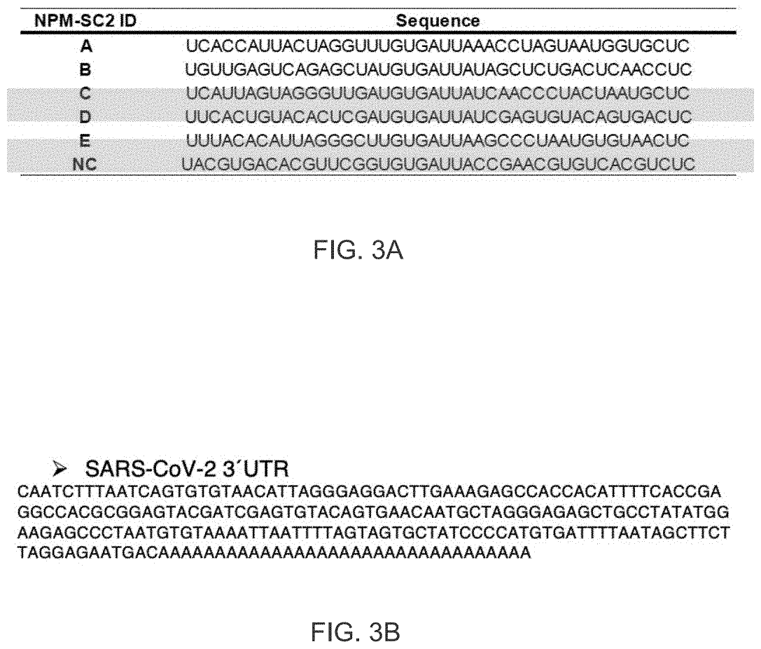

Thus, a series of RNAi-inducing siRNA/miRNA molecules may be designed to specifically target the SARS-CoV-2 viral RNA at different locations. The sequences shown in FIG. 3A may have been synthesized, and may have obtained results for the first candidate, labeled as “D”. The scrambled, non-effective sequence “NC” may be used as a “negative control.”

Sequence “D” may be tested for efficacy against a luciferase reporter construct, designed to include the 3′UTR (untranslated region) of the SARS-CoV-2 RNA, as shown in FIG. 3B.

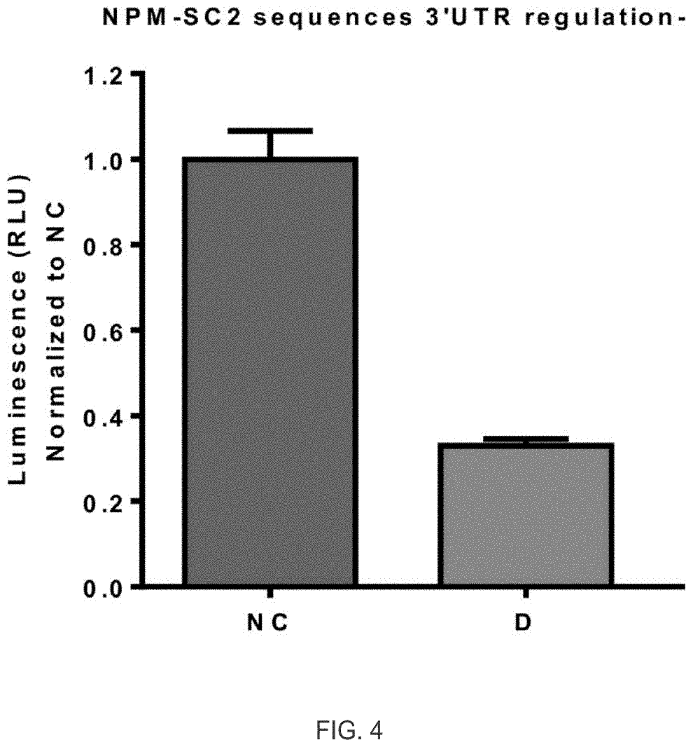

293T cells may be transfected with both the luciferase reporter construct and the antiviral sequence “D,” as described above. Following forty-eight hours, the luciferase expression of the reporter construct may be examined using a plate reader. There may be a significant reduction in luciferase activity following transfection of sequence “D,” demonstrating the regulatory potential and efficacy of the designed sequence. A non-limiting example of the results is shown in FIG. 4.

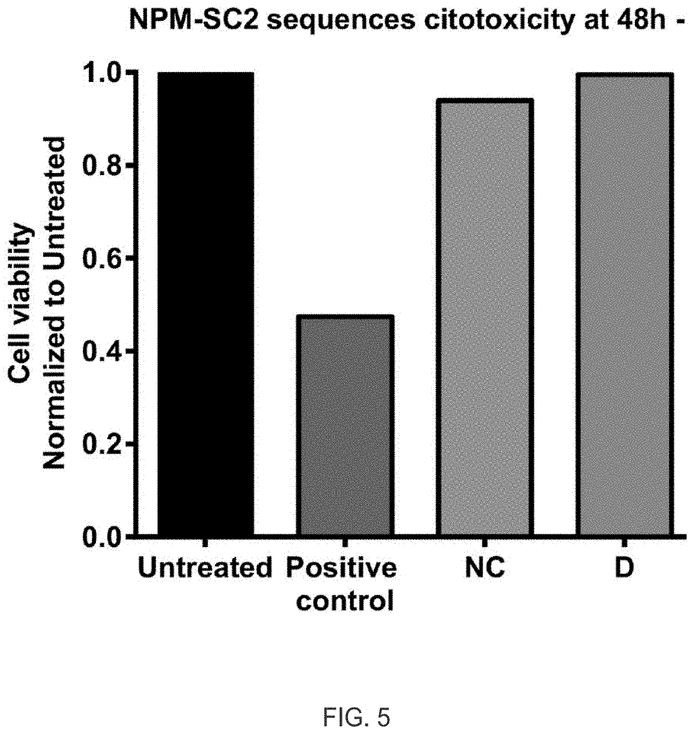

Potential cytotoxic effects of sequence “D” in fibroblasts may also be tested. The cells may be transfected with either a positive cytotoxic control, the NC, or sequence “D.” In an embodiment, no cytopathic effects may be observed through MTS assay at 48 hours post transfection, indicating the relative safety of the sequence. A non-limiting example of the results is shown in FIG. 5.

In an embodiment, the invention of the present disclosure may be a method for identifying an RNAi sequence, comprising the steps of identifying a potential 7-mer sequence; and registering one or more hit sites, where the one or more hit sites are one or more positions in a genomic RNA sequence where the potential 7-mer sequence is present. In an embodiment, the method may further comprise the steps of registering, alongside with the one or more hit sites, a name of the genomic region where the 7-mer sequence was found and a registered 15-mer sequence starting at a 7-mer sequence start position; generating, using the registered 15-mer sequence, a nucleotide position frequency matrix and a generated 15-mer for each 7-mer sequence based on the most frequent nucleotide for each position; and predicting, using the generated 15-mer sequence, one or more hits for a putative RNAi molecule. In an embodiment, the method may also comprise the steps of calculating a hit feasibility index for the one or more registered hit sites; generating a general index; and developing a summary matrix.

In an embodiment, the potential 7-mer sequence may be identified by screening for the most 7-mers in a target RNA. In an embodiment, the one or more hits for the putative RNAi molecule may be predicted based on perfect matching. In an embodiment, the hit feasibility index may assign a weight to each hit site based on a match sequence ΔG value and an AU content proportion 30 nucleotides upstream and downstream from the hit site. The hit feasibility index may be calculated via the following equation: Hₙ_{ₘ}=log₃(p(AU)ₙ_{ₘ}×−ΔGₙ_{ₘ}), where n is the generated 15-mer used to predict a m hit site, H is the hit feasibility, ΔG is the free energy required for the match to happen considering only base pairing, and p(AU) is the proportion 30 nt upstream and downstream from the hit site.

In an embodiment, the general index may be calculated by the following equation: IGₘ=Σₖ₌₁(Σₘ₌₁ Hₙ_{ₘ}), where IG is the general index which summarizes the effect of the hits, and A is the amount of hits for the n generated 15-mer of the match. The summary matrix may comprise the 7-mer, the 7-mer's generated 15-mer, a sense strand, the RNAi proposed sequence, the hit feasibility index, and the general index. In a further embodiment, the summary matrix may comprise a proposed antisense strand. In yet another embodiment, the summary matrix may further comprise a seed sequence GC content and a guide strand GC content.

In an embodiment, the invention of the present disclosure may be a method for identifying an RNAi sequence, comprising the steps of screening for one or more 7-mers from a gRNA; selecting a first number of the one or more 7-mers, the first number based on the frequency of the one or more 7-mers; and characterizing one or more hit sites for each of the one or more 7-mers. The method may further comprise the steps of calculating a hit per genomic region for each of the one or more 7-mers; selecting a second number of the one or more 7-mers, the second number based on the most hits in a sgRNA; and creating a frequency matrix for one or more 15-mers associated to each of the one or more 7-mers. In a further embodiment, the method also includes the steps of generating, based on the most frequent nucleotides, one or more generated 15-mers for each of the one or more 7-mers; characterizing hits in the gRNA for each of the one or more generated 15-mers; calculating a summary index based on a cumulative hit frequency and a length; and selecting a third number of the RNAi sequences based on one or more generated features and one or more structural features.

In an embodiment, the first number, the second number, and the third number may be any appropriate quantities. In an embodiment, the first number may be 500. In an embodiment, the second number may be 50. In an embodiment, the third number may be 5. In an embodiment, the one or more generated features and the one or more structural features may be a function of the summary index.

While this invention has been described in conjunction with the embodiments outlined above, many alternatives, modifications and variations will be apparent to those skilled in the art upon reading the foregoing disclosure. For example, the aforementioned embodiments may include specific quantities, chemicals, compounds, genetic material, sequences, time periods, or other quantities. In various embodiments, any of the aforementioned quantities may be non-limiting. Accordingly, the embodiments of the invention, as set forth above, are intended to be illustrative, not limiting. Various changes may be made without departing from the spirit and scope of the invention.

What is claimed is:

1. A method for identifying an RNAi sequence, comprising the steps of:
a. identifying a potential 7-mer sequence;
b. registering one or more hit sites, wherein the one or more hit sites are one or more positions in a genomic RNA sequence where the potential 7-mer sequence is present;
c. registering, alongside with the one or more hit sites, a name of the genomic region where the 7-mer sequence was found and a registered 15-mer sequence starting at a 7-mer sequence start position;
d. generating, using the registered 15-mer sequence, a nucleotide position frequency matrix and a generated 15-mer for each 7-mer sequence based on the most frequent nucleotide for each position;
e. predicting, using the generated 15-mer sequence, one or more hits for a putative RNAi molecule;
f. calculating a hit feasibility index for the one or more registered hit sites;
g. generating a general index; and
h. developing a summary matrix.

2. The method of claim 1, wherein the potential 7-mer sequence is identified by screening for the most 7-mers in a target RNA.

3. The method of claim 1, wherein the one or more hits for the putative RNAi molecule are predicted based on perfect matching.

4. The method of claim 1, wherein the hit feasibility index assigns a weight to each hit site based on a match sequence ΔG value and an AU content proportion 30 nucleotides upstream and downstream from the hit site.

5. The method of claim 4, wherein the hit feasibility index is calculated via the following equation:
Hₙ_{ₘ}=log₃(p(AU)ₙ_{ₘ}×−ΔGₙ_{ₘ}),
wherein n is the generated 15-mer used to predict a m hit site, H is the hit feasibility, ΔG is the free energy required for the match to happen considering only base pairing, and p(AU) is the proportion 30 nt upstream and downstream from the hit site.

6. The method of claim 5, wherein the general index is calculated by the following equation:
IGₘ=Σₖ₌₁(Σₘ₌₁Hₙ_{ₘ}),
wherein IG is the general index which summarizes the effect of the hits, and A is the amount of hits for then generated 15-mer of the match.

7. The method of claim 6, wherein the summary matrix comprises the 7-mer, the 7-mer's generated 15-mer, a sense strand, the RNAi proposed sequence, the hit feasibility index, and the general index.

8. The method of claim 7, wherein the summary matrix further comprises a proposed antisense strand.

9. The method of claim 7, wherein the summary matrix further comprises a seed sequence GC content and a guide strand GC content.

10. A method for identifying an RNAi sequence, comprising the steps of:
a. screening for one or more 7-mers from a gRNA;
b. selecting a first number of the one or more 7-mers, the first number based on the frequency of the one or more 7-mers;
c. characterizing one or more hit sites for each of the one or more 7-mers;
d. calculating a hit per genomic region for each of the one or more 7-mers;
e. selecting a second number of the one or more 7-mers, the second number based on the most hits in a sgRNA;
f. creating a frequency matrix for one or more 15-mers associated to each of the one or more 7-mers;
g. generating, based on the most frequent nucleotides, one or more generated 15-mers for each of the one or more 7-mers;
h. characterizing hits in the gRNA for each of the one or more generated 15-mers;
i. calculating a summary index based on a cumulative hit frequency and a length; and
j. selecting a third number of the RNAi sequences based on one or more generated features and one or more structural features.

11. The method of claim 10, wherein the first number is 500.

12. The method of claim 10, wherein the second number is 50.

13. The method of claim 10, wherein the third number is 5.

14. The method of claim 10, wherein the one or more generated features and the one or more structural features are a function of the summary index.
